(19) United States
(12) Patent Application Publication
(10) Pub. No.: US 2018/0214273 A1
(43) Pub. Date: Aug. 2, 2018

(54) JOINT IMPLANT COMPONENT HAVING A FIXATION STRUCTURE FOR SOFT TISSUE

(71) Applicant: Waldemar Link GmbH & Co. KG, Hamburg (DE)

(72) Inventor: Helmut D. Link, Hamburg (DE)

(73) Assignee: Waldemar Link GmbH & Co. KG, Hamburg (DE)

(21) Appl. No.: 15/886,878

(22) Filed: Feb. 2, 2018

(30) Foreign Application Priority Data
Feb. 2, 2017 (DE) 10 2017 201 666.3

(51) Int. Cl. A61F 2/32; A61B 17/04

(52) U.S. Cl. CPC A61F 2/32; A61F 2/38; A61B 17/0401

(57) ABSTRACT

The present invention provides a joint implant component as well as a method for the implantation thereof. The joint implant component (1), in particular of a hip endoprosthesis, has a proximal joint section (10), a distal anchoring section (30) and a transition section (20) having a fixation structure (21) between the joint section (10) and the anchoring section (30), the fixation structure being arranged in at least one part of the perimeter of the transition section (20) as an elongate recess (22) for guiding suture material.

### FIELD OF THE INVENTION

The present invention relates to a joint implant component having a fixation structure for attaching soft tissue via suture material and a method for fixing soft tissue via the fixation structure to the joint endoprosthesis.

### PRIOR ART

When implanting a joint endoprosthesis, the implantation site must first be prepared for the implantation of at least one joint implant component. The joint that is to be replaced, which can be a native or also an already-present artificial joint, is removed and the bone tissue is adjusted for receiving the joint implant component.

During the operation, an access to the joint is first made in order to be able to prepare the bone tissue for receiving the joint implant component. Here, soft tissue is separated from the part of the bone which is to be treated for the preparation of the implantation site. In particular, tendons of muscle insertions can be among this soft tissue. This separation from soft tissue particularly occurs in revision surgeries, i.e. operations in which an already present joint implant is replaced by a new one. As a result thereof, a loss of stability around the joint can occur.

Consequently, it is desirable to re-attach such soft tissue which has been separated from the bone tissue in order to achieve a restoration that is as comprehensive as possible of the bio-mechanical functionality of the joint to be replaced. Different techniques for attaching to bone tissue are already known here, which mostly use bone screws in order to anchor soft tissue via suture material to a bone. Such bone screws are disclosed, for example, in DE 10 2011 016 659 Al and DE 10 2013 206 367 A1.

However, these devices are not suited for anchoring suture material to a joint implant component since a notch effect can occur at a hole introduced into the joint implant component, which possibly has a negative effect on the stability of the joint implant component. This is particularly the case if a screw hole for receiving an anchoring screw is introduced into the shaft of the joint implant component since this is subjected to a dynamic load by the joint forces acting on it. Moreover, additional securing means are required when using an anchoring screw in order to secure it against an unintentional detachment of the anchoring screw.

A further technique for attaching soft tissue is the use of meshes which are laid around the part of the joint implant component to which the soft tissue is to be attached. Such a mesh is proposed, for example, in DE 10 2010 007 707 A1. However, these meshes must in turn be attached to bone tissue or the joint implant component. The latter is only possible by precisely adapting the mesh to the shape of the prosthesis so that it lies around the outer design of the prosthesis like a stocking. It is necessary here that a sufficiently large part of the prosthesis is exposed, around which the mesh can be laid.

### SUMMARY OF THE INVENTION

Accordingly, the present invention is based on the object of providing an attaching possibility for soft tissue to a joint implant component by means of which the soft tissue can be attached for a restoration of the bio-mechanical functionality that is as comprehensive as possible. Additionally, it was the object of the invention to provide a method with which the soft tissue can be attached to the joint implant component.

This object is solved by the joint implant component defined in the independent claim 1 and the method for implanting a joint implant component in claim 11. Additionally, the independent claims define preferred embodiments of the invention.

Thus, the invention provides a joint implant component, in particular of a hip endoprosthesis, which has a proximal joint section, a distal anchoring section and a transition section having a fixation structure between the joint section and the anchoring section, the fixation structure being arranged in at least one part of the perimeter of the transition section as an elongate recess for guiding attaching material, in particular suture material.

A movement of the attaching material along the longitudinal direction of the joint implant component is prevented by the elongate recess for guiding attaching material. In other words, the sides of the elongate recess facing one another limit the freedom of movement of the attaching material.

The attaching material serves the attachment of soft tissue. All kinds of material suited for use in the human body and with which an open or closed loop can be formed in the peripheral direction of the implant component and can be guided along the elongate recess are eligible as an attaching material. It is particularly possible to use suture material as loop material, however, other materials such as, for example, wire, a clasp or a ring are conceivable as well.

The elongate recess accordingly extends in the peripheral direction, i.e. at an angle to the longitudinal direction of the joint implant component. Such a joint implant component can be, for example, an implant shaft such as, for example, an implant shaft of a hip, shoulder, or knee prosthesis.

As further set out below, the suture material can, for example, be used for attaching soft tissue in which it is guided through the elongate recess of the joint implant component, by which soft tissue is sewn and a loop around the perimeter of the implant component is formed. Thus, when fixed such the soft tissue is secured in the longitudinal direction of the joint implant component in the manner already described above.

In a preferred embodiment, the fixation structure of the transition section comprises a soft tissue contact area which is preferably substantially opposite to the recess.

Such a soft tissue contact area works together with the elongate recess and thereby improves the connection between the soft tissue and the implant component. More precisely, a relative movement between the soft tissue and the implant surface is prevented by the soft tissue contact area. Not only does the soft tissue contact area here preferably counteract a movement in the longitudinal direction of the implant component, but rather, a movement in the peripheral direction as well.

Preferably, the soft tissue contact area is essentially diametrically opposite to the elongate recess. It is also possible to provide such a soft tissue contact area as part of the fixation structure on the entire perimeter of the transition section. However. the elongate recess is preferably excluded therefrom so that it is provided to keep the soft tissue outside of the elongate recess on the joint implant component.

In one particularly preferred embodiment, the soft tissue contact area has a coating.

A coating has the advantage that a relative movement between the soft tissue and the surface of the joint implant component can be especially effectively prevented by it. The coating, for example, has a surface structure therefor which leads to an anchoring of the soft tissue. Such an anchoring is achieved, in particular, by a growing onto or growing into the surface structure.

In one further preferred embodiment, the recess at its ends in the longitudinal direction continuously transitions into the outer surface of the transition section.

In this embodiment, the elongate recess extends around one part of the perimeter. Due to the continuous transition to the outer surface, there are no edges in the area into which the coating material extends so that a possible damaging of the coating material by a relative movement between it and the surface of the joint implant component is counteracted.

In one preferred embodiment, the fixation structure has two, three or four elongate recesses for guiding the attaching material.

This embodiment enables an advantageous adaptation of the fixation structure to the anatomical conditions in the surroundings of the joint implant component so that an arrangement which is as functional as possible of the soft tissue at the joint implant component and in particular at its perimeter can be achieved.

In this embodiment, the plurality of elongate recesses can not only be arranged at an angle to the longitudinal axis of the joint implant component, but rather also relative to one another.

In one further embodiment, the elongate recess extends over a range of at least 45° and preferably at least 90° up to a maximum of 270° and preferably 180° around the perimeter of the transition section.

Within these angle ranges, a sufficient resistance against a shifting of the coating material in the longitudinal direction is provided and the irregularity of the implant geometry is kept as low as possible, by which stress peaks in the implant component are avoided.

In one further embodiment of the joint implant component, the cross-section profile of the recess is a continuous and preferably curved profile.

Owing to such a cross-section profile, stress peaks in the stress distribution are reduced by the joint implant component and thus an effect on the elongate recess on the joint implant component is kept low.

Since the profile is carried out continuously, it has no sudden transitions at which stress peaks can occur. This also allows for the absence of edges in the elongate recess which could, under circumstances, lead to a damaging of the attaching material.

The continuously carried out cross-section profile of this embodiment can comprise straight sections which transition into curved sections or also preferably only curved sections.

In one further embodiment the cross-section profile of the elongate recess has a width of at least 2 mm, preferably at least 3 mm, and more preferably at least 4 mm to a maximum of 12 mm, preferably a maximum of 10 mm and more preferably a maximum of 8 mm.

Independently of the configuration of the cross-section profile, such a width of the elongate recess reduces the influence of this recess on the geometry of the implant component and thereby reduces the distribution of stress. Here, the lower values provide a lower freedom of movement for the attaching material while the upper values enable a rather more fluid and thus an unobtrusive contour change of the implant shape. Additionally, due to their size-dependent accesibility, the upper values are easier for a surgeon to handle.

Independently of the other parameters of the previous two embodiments having the lower values, the depth of the cross-section profile provides a lower alteration of the implant surface with the lower values, and better conditions for holding the coating material with the higher values.

In one further preferred embodiment, the joint implant component is a hip endoprosthesis, in particular a shaft of a hip endoprosthesis, the middle point or a middle area of the recess being in the longitudinal direction thereof on the medial side of the hip endoprosthesis. The recess is furthermore preferably formed or arranged symmetrically to the frontal plane.

In this embodiment, the elongate recess serves to fix soft tissue to the lateral side of the hip endoprosthesis. It is provided here that the soft tissue is located in the peripheral direction substantially on the side of the prosthesis shaft that is approximately opposite to the middle point of the recess. The greater trochanter which serves as the attachment for the gluteal muscle is located at approximately this point in a native hip joint and a native femur.

Furthermore, the present invention provides a method for the implantation of a joint implant component as described above. Here, the method comprises the steps: preparing a cavity in the bone tissue for receiving an anchoring portion of the joint implant component; inserting the joint implant component into the cavity so that the fixation structure of the transition section of the joint implant component is outside of the cavity; connecting soft tissue which is to be attached to the joint implant component with an attaching material; placing the attaching material along the peripheral direction of the joint implant component in the elongate recess of the fixation structure; narrowing the attaching material in the elongate recess so that it cannot move out of the elongate recess in the longitudinal direction of the joint implant component.

As a result of this method, the soft tissue at the joint implant component is held and thereby regains bio-mechanical functionality.

The connection of the soft tissue with the attaching material can occur here directly or indirectly. When attaching directly, the soft tissue is connected with the attaching material and preferably arranged at least in sections between the attaching material and the joint implant component. In the case of an indirect attachment, the attaching material serves to attach a further attaching means thereto for fixing the soft tissue.

In one preferred embodiment of the method, the attaching material is a suture material which is sewn together with the soft tissue and then placed in the elongate recess of the fixation structure.

With the suture material, it is possible to produce the connection in the proven manner with soft tissue via a suture. Here, this suture is preferably carried out such that the soft tissue cannot move along the suture material. Consequently, it is a direct attachment of the soft tissue to the attaching material. After stitching the soft tissue, the suture material can then form a loop around the perimeter of the implant component which once placed in the elongate recess of the joint implant component, is pulled together and thereby narrowed.

Alternatively or additionally to the previous embodiment, in one further embodiment of the method the coating material can be a wire or a ring.

A connection of the soft tissue with the joint implant component can also be produced with a wire or a ring. Such a connection has the advantage that it can be carried out stronger, narrower and/or more accurately fitting than with suture material, and consequently further restricts a relative movement between the attaching material and the joint implant component. By contrast, suture material can be used more flexibly.

Furthermore, with the attachment material of this embodiment an indirect attaching of the soft tissue to the ring or wire is preferred. In order for it to be narrowed at the height of the elongate recess, the ring can, for example, be carried out as an elastic C-ring or also as a mountable ring. A mountable ring is preferably formed here by at least two partial pieces.

In one further embodiment of the method, the soft tissue to be attached is arranged on a soft tissue contact area which is preferably arranged in the peripheral direction outside of the recess and more preferably approximately on the opposite side of the recess.

Above all, the placement of the soft tissue on a soft tissue contact area which is particularly carried out as already stated above raises the stability of the connection between the soft tissue and the joint implant component.

### BRIEF DESCRIPTION OF THE FIGURES

The following figures illustrate a preferred embodiment of the present invention. This embodiment is not intended to restrict the scope of protection of the claims, but rather together with the description only serves to simplify an understanding of the invention.

FIG. 1 is a view of the frontal plane of a preferred embodiment of a joint implant component which has a fixation structure for attaching soft tissue.

FIGS. 2a and 2b are schematic illustrations of possible cross-sections of the elongate recess.

### DETAILED DESCRIPTION OF PREFERRED EMBODIMENTS

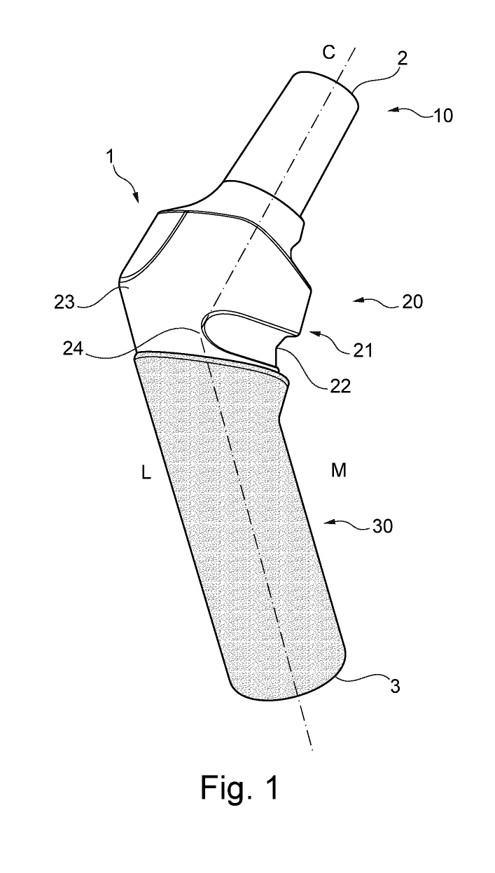

FIG. 1 shows a joint implant component 1. This exemplary embodiment relates here to a joint implant component of a hip joint endoprosthesis, in particular the implant shaft for an anchoring in the bone tissue of a patient. Other joints in which the present invention can be implemented are, as already stated above, for example, a knee joint component or a shoulder joint component.

The joint implant component 1 has a joint section 10 and an anchoring portion 30. The joint section 10 is located on the proximal side 2 of the joint implant component 1, while the anchoring section 30 is arranged on the distal side 3 of the joint implant component 1. A transition section 20 is arranged between the joint section 10 and the anchoring section 30.

As shown in FIG. 1, the joint section 10 can be on a side of a cone connection, with which further joint implant components such as, for example, a joint head which is not shown can be connected. The anchoring section 30 is provided to anchor the joint implant component 1 to the bone tissue. The anchoring section 30 of the joint implant component 1 that is shown in FIG. 1 is configured, for example, such that the surrounding bone tissue grows into the surface structure of the anchoring section 30 and thus anchors the joint implant component 1 into the bone.

However, the anchoring section 30 can also be carried out differently, such as for example, smooth or polished in order to anchor the joint implant component 1 in a bone with bone cement. Moreover, further attaching elements or other attaching elements such as, for example, bone screws inter alia can be used for the anchoring.

In the transition section 20, there is a fixation structure 21 which in turn has an elongate recess 22 for receiving attaching material. As shown in FIG. 1, the elongate recess 22 extends across at least one part of the perimeter of the joint implant component 1. Moreover, the elongate recess or groove 22 is formed perpendicularly or at at an angle to the longitudinal axis C of the joint implant component 1 extending on the outer side thereof. The recess 22 preferably extends here in a plane.

It is also possible to form the elongate recess 22 across the entire perimeter of the joint implant component 1. However, it is preferred to only form the recess 22 across one part of the perimeter. It is especially advantageous here if the ends 24 of the elongate recess 22 are continuously tapered, i.e. that the depth of the recess 22 continuously decreases towards its ends 24. As described above it is thereby ensured by the elongate recess 22 that there are no edges or ledges which could damage the attaching material engaging into the recess 22.

The elongate recess 22 of the joint implant component 1 shown in FIG. 1 is provided on the medial side M of the joint implant component 1. It serves to hold soft tissues, in particular ligaments, tendons and muscle insertions on the lateral side L, i.e. the side of the implant 1 which is substantially opposite to the elongate recess 22, with the help of the attaching material. In other words, there is a soft tissue contact area 23 at a recess on the medial side M, preferably on the lateral side L.

As shown in the exemplary embodiment in FIG. 1, this soft tissue contact area 23 can have the same surface finish as the elongate recess 22.

However, with one modification it is equally possible to provide the soft tissue contact area 23 with a surface having a structure and/or properties which are configured for a connection with soft tissue. Such a surface particularly has the advantage that it restricts the range of movement of the soft tissue along the longitudinal direction of the implant component 1 and in the peripheral direction.

Furthermore, the fixation structure 21 can have at least one surface in sections which counteracts an ingrowth of bone tissue. This is particularly advantageous for the soft tissue contact area 23. By contrast, as to the elongate recess 22 it can be advantageous that bones grow into the surface in order to thus prevent a movement of the attaching material. In order to achieve this, the surface of the elongate recess 22 can at least in sections be provided with a surface structure which is configured for the bone tissue to grow into.

As described above, the maximum width B of the cross-section profile of the elongate recess 22 is preferably at least 2 mm, preferably at least 3 mm and more preferably at least 4 mm and a maximum of 12 mm, preferably a maximum of 10 mm and more preferably a maximum of 8 mm.

The depth T of the cross-section of the elongate recess 22 preferably is from at least 2 mm, preferably 3 mm to a maximum of 8 mm and preferably a maximum of 6 mm.

It generally applies that the smaller the depth and the wider the width B of the elongate recess 22 is in its cross-section, the less the elongate recess 22 affects the distribution of stress through the joint implant component 1. These stresses are caused by the transmission of the joint forces into the bones. By contrast, it is advantageous for holding the attaching material in the elongate recess 22 if the depth T of the elongate recess 22 is as large as possible since it thereby becomes less likely that the attaching material moves out of the recess perpendicular to the longitudinal direction of the elongate recess 22. The above locations each provide a width B and a depth T for the elongate recess 22, in which a reliable holding of the attaching material in the recess 22 is enabled without significantly influencing the distribution of stress in the joint implant component 1.

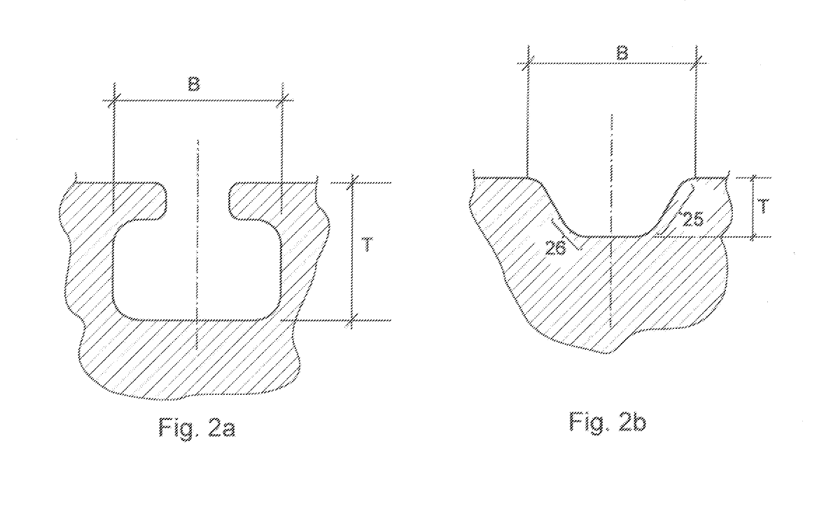

Regarding the shape of the cross-section of the elongate recess 22, in particular, a continuous extension of said cross-section is preferred in order to thus prevent sudden transitions in the cross-section which would lead to the formation of edges in the elongate recess 22. An exemplary embodiment of a cross-section extension is illustrated in FIG. 2a, which is formed from straight sections, such as the straight section 25, and curved sections, such as the curved section 26. More preferably, the cross-section profile of the elongate recess 22 consists of continuously extending curved sections.

The cross-section profile of the elongate recess 22 preferably has a substantially, continuously decreasing width B with an increasing depth T, for example, as with a V-shaped course. A V-shaped course here has the advantage that the tip of the cross-section, i.e. the bottom of the cross-section does not have any negative effects on the attaching material. Here, the tip of the cross-section is preferably provided with a radius, i.e. rounded.

It is also possible to use a cross-section extension, the width B thereof increases in sections with increasing depth T, before the bottom of the cross-section or the elongate recess 22 is reached (see FIG. 2b). A recess 22 is provided thereby, the side walls thereof additionally counteract a sliding out of the attaching material in the longitudinal direction. In other words, such a cross-section forms a cavity which has a narrowing at its entrance side.

As shown in FIGS. 2a and 2b, a continuous transition with the cross-section profile of the elongate recess 22 can also be provided between the side walls of the elongate recess and the outer surface or surface area of the implant.

In addition, the fixation structure 21 provided by the joint implant component according to the invention, the implant component is particularly suited for revision, since with it there is more soft tissue to be removed compared with a first treatment.

### Reference Numbers

1 Joint implant component
2 Proximal side
3 Distal side
10 Joint portion
20 Transition section
21 Fixation structure
22 Elongate recess of the attaching material receiver
23 Soft tissue contact area
24 Recess end
25 Straight section
26 Curved section
30 Anchoring section
C Longitudinal axis
L Lateral side
M Medial side

1-14. (canceled)

15. Joint implant component (1), in particular of a hip endoprosthesis, which comprises a proximal joint section (10), a distal anchoring section (30) and a transition section (20) having a fixation structure (21) between the joint section (10) and the anchoring section (30), the fixation structure (21) being arranged in at least one part of the perimeter of the transition section (20) as an elongate recess (22) for guiding suture material.

16. Joint implant component (1) according to claim 15, in which the fixation structure (21) of the transition section (20) comprises a soft tissue contact area (23) which is preferably substantially opposite to the recess (22).

17. Joint implant component (1) according to claim 16 in which the the soft tissue contact area comprises a coating.

18. Joint implant component (1) according to claim 15, in which the recess (22) at its ends (24) in the longitudinal direction continuously transitions into the outer surface of the transition section (20).

19. Joint implant component (1) according to claim 16, in which the recess (22) at its ends (24) in the longitudinal direction continuously transitions into the outer surface of the transition section (20).

20. Joint implant component (1) according to claim 17, in which the recess (22) at its ends (24) in the longitudinal direction continuously transitions into the outer surface of the transition section (20).

21. Joint implant component (1) according to claim 15, in which the fixation structure (21) comprises one, two, three or four elongate recesses (22) for guiding the attaching material.

22. Joint implant component (1) according to claim 16, in which the fixation structure (21) comprises one, two, three or four elongate recesses (22) for guiding the attaching material.

23. Joint implant component (1) according to claim 15, in which the elongate recess (22) extends in an area of at least 45° and preferably at least 90° up to a maximum of 270° and preferably 180° around the perimeter of the transition section (20).

24. Joint implant component (1) according to claim 15, in which the cross-section profile of the elongate recess (22) is a continuous and preferably curved profile.

25. Joint implant component (1) according to claim 15, in which the cross-section profile of the elongate recess (22) has a width of at least 2 mm, preferably at least 3 mm and more preferably at least 4 mm up to a maximum of 12 mm, preferably a maximum of 10 mm and more preferably a maximum of 8 mm.

26. Joint implant component (1) according to claim 15, in which the cross-section profile of the recess (22) has a depth of at least 2 mm, preferably at least 3 mm up to a maximum of 8 mm and preferably a maximum of 6 mm.

27. Joint implant component (1) according to claim 15, wherein the joint implant component (1) is a hip endoprosthesis and the middle point in the longitudinal direction of the recess (22) is on the medial side of the hip endoprosthesis and is preferably arranged symmetrically to the frontal plane.

28. Joint implant component (1) according to claim 16, wherein the joint implant component (1) is a hip endoprosthesis and the middle point in the longitudinal direction of the recess (22) is on the medial side of the hip endoprosthesis and is preferably arranged symmetrically to the frontal plane.

29. Joint implant component (1) according to claim 17, wherein the joint implant component (1) is a hip endoprosthesis and the middle point in the longitudinal direction of the recess (22) is on the medial side of the hip endoprosthesis and is preferably arranged symmetrically to the frontal plane.

30. Joint implant component (1) according to claim 18, wherein the joint implant component (1) is a hip endoprosthesis and the middle point in the longitudinal direction of the recess (22) is on the medial side of the hip endoprosthesis and is preferably arranged symmetrically to the frontal plane.

31. Method for implanting a joint implant component (1) according to claim 15, wherein the method comprises the steps:
a. preparing a cavity in the bone tissue for receiving the anchoring section (30) of the joint implant component (1);
b. inserting the joint implant component (1) into the cavity so that the fixation structure (21) of the transition section (20) of the joint implant component (1) is outside of the cavity;
c. connecting soft tissue which is to be attached to the joint implant component with an attaching material;
d. placing the attaching material along the peripheral direction of the joint implant component (1) in the elongate recess (22) of the fixation structure (21);
e. narrowing the attaching material in the elongate recess so that it cannot move out of the elongate recess (22) in the longitudinal direction of the joint implant component.

32. Method for implanting a joint implant component (1) according to claim 31, wherein the attaching material is a suture material which is sewn together with the soft tissue before it is placed in the elongate recess of the fixation structure.

33. Method for implanting a joint implant component (1) according to claim 31, wherein the attaching material is a wire or a ring.

34. Method for implanting a joint implant component (1) according to claim 31, wherein the soft tissue to be attached is arranged on a soft tissue contact area (23) that is preferably outside of the recess (22) in the peripheral direction and more preferably is arranged approximately on the opposite side of the recess.
